(19) United States
(12) Patent Application Publication
(10) Pub. No.: US 2026/0344997 A1
(43) Pub. Date: Nov. 5, 2026

(54) APPARATUS AND METHOD FOR COLLECTING DATA FOR DETERMINING A REFRACTIVE ERROR

(71) Applicant: IBISVISION LTD., Glasgow (GB)

(72) Inventors: Jason Higginbotham, Glasgow (GB); Peter Estibeiro, Glasgow (GB)

(73) Assignee: IBISVISION LTD., Glasgow (GB)

(21) Appl. No.: 18/997,644

(22) PCT Filed: Jul. 21, 2023
(86) PCT No.: PCT/EP2023/070375

§ 371 Date: Jan. 22, 2025

(30) Foreign Application Priority Data
Jul. 22, 2022 (GB) 2210797.3
Sep. 13, 2022 (GB) 2213395.3
Jul. 18, 2023 (GB) 2311010.9

(51) Int. Cl. A61B 3/032

(52) U.S. Cl. CPC A61B 3/032

(57) ABSTRACT

Apparatus for use in collecting data for determining a myopic or hyperopic sphero-cylindrical refractive error of a human eye including cylinder axis and near addition. The apparatus comprises a Scheiner component comprising first and second spaced apart apertures, and orientation indicia on or adjacent to the Scheiner component for allowing a rotational position of the apertures relative to a user's eye to be determined when the apparatus is in use. A method and a target for use with such an apparatus are also described.

### FIELD OF THE INVENTION

This invention pertains generally to the field of optometry.

### BACKGROUND OF THE INVENTION

Optometrists use a variety of optometric instruments to estimate refractive errors of the human eye. Examples of such refractive errors include spherical error (e.g., myopia or hyperopia) and cylindrical error (e.g., astigmatism). Such instruments are often expensive and require significant training to operate.

### SUMMARY OF THE INVENTION

In accordance with a first aspect, there is provided an apparatus for use in collecting data for determining a myopic or hyperopic sphero-cylindrical refractive error of a human eye including cylinder axis and near addition, the apparatus comprising:
a Scheiner component comprising first and second spaced apart apertures; and
orientation indicia on or adjacent to the Scheiner component for allowing a rotational position of the apertures relative to a user's eye to be determined when the apparatus is in use.

The orientation indicia may comprise rotational indicia on the Scheiner component.

The apparatus may comprise a frame, the Scheiner component being mounted for rotation relative to the frame.

The Scheiner component may comprise at least first, second, and third pairs of the first and second spaced apart apertures, the first, second, and third pairs being spaced apart from each other on the Scheiner component.

The apparatus may be configured for use with the Scheiner component in each of a plurality of positions, each of the positions corresponding with one of the at least first, second, and third pairs of apertures being in a viewing position, wherein, when at the viewing position, each of the first, second, and third pairs of the apertures has its first and second apertures aligned at a different angle as compared with an angle of alignment of the other of the first, second, and third pairs at the viewing position.

The apparatus may comprise a frame, the Scheiner component being mounted to the frame for movement between each of the plurality of positions.

The Scheiner component may be mounted to the frame for rotation between each of the plurality of positions.

The Scheiner component may be mounted to the frame for translational movement between each of the plurality of positions.

The frame may comprise a window that defines the viewing position, the apparatus being configured such at each of the plurality of positions, a different one of the first, second, and third pairs appears through the window.

The orientation indicia may comprise frame indicia on the frame. The frame indicia may comprise a scale, which may optionally be configured for allowing a distance from an image capture device to the frame to be determined by a processor in communication with the image capture device.

The frame may comprise a handle that can be grasped to hold the apparatus in front of a user's eye when the apparatus is in use.

The frame may comprise a bridge-engaging portion for engaging a bridge of a user's nose when the apparatus is in use.

The apparatus may comprise a positive power lens for generating a positive refractive offset. The positive power lens may be removably mountable to the apparatus.

The apparatus may comprise a lens mount for mounting the lens.

The lens mount may be moveable between a first position in which the lens is in front of a user's eye when the apparatus is in use, and a second position in which the lens is not in front of the user's eye when the apparatus is in use. The lens mount may be mounted such that it can be moved between the first position and the second position.

In accordance with a second aspect, there is provided a target for use with an apparatus having a Scheiner component for use in collecting data for determining a myopic or hyperopic sphero-cylindrical refractive error of a human eye including cylinder axis and near addition, the Scheiner component comprising a pair of apertures that are spaced apart along a first axis, the target comprising a first feature for determining a correspondence between a presentation angle of the first feature and the first axis.

The first feature may be elongate.

The first feature may comprise at least one linear component.

The linear component may comprise a first line.

The target may include a second feature for determining, using the Scheiner component of the apparatus, a first far point of the human eye by adjusting a distance between the human eye and the target.

The first feature and the second feature may be elongate in orthogonal directions relative to each other.

The second feature may comprise at least one linear component.

The linear component may comprise at least one second line.

The first and second features may together comprise one or more crosses, grids, shapes, and/or sets of linear components.

The first feature may comprise at least one line and the second feature may comprise a line extending orthogonally to the line of the first feature.

The first feature and the second feature may be visually distinct from each other.

The target may be rotatable.

The target may be rotatable between first and second orthogonal rotational positions.

The target may be rotatable between first, second, and third rotational positions, which may optionally be equidistant.

The target may comprise indicia for determining a rotational position of the target.

In accordance with a third aspect, there is provided a method of collecting data for determining a sphero-cylindrical refractive error of a human eye, the method comprising:
determining a rotational angle of a first cylindrical axis of an eye for which data is to be collected;
determining, based on the Scheiner principle, a first far point of the eye for the first cylindrical axis using the apparatus of the preceding aspect; and
determining, based on the Scheiner principle, a second far point of the eye for a second cylindrical axis using the apparatus, with the Scheiner component orientated orthogonally to an angle at which the first far point was determined, wherein a rotational angle of the second cylindrical axis is orthogonal to the rotational angle of the first cylindrical axis.

The method may comprise:
determining whether a refractive error of an eye for which data is to be collected is greater than a predetermined value; and
responsive to determining that the refractive error of the eye is greater than the predetermined value, positioning a positive power lens relative to the apparatus such that it is in front of a user's eye when the apparatus is in use, prior to determining the first and second far points.

The predetermined value may be less than 0.0 D, or optionally less than −1.00 D.

Positioning the positive power lens may comprise installing the lens on a lens mount of the apparatus.

Positioning the positive power lens may comprise moving the positive power lens from a second position in which the lens is not in front of the user's eye when the apparatus is in use, to a first position.

In accordance with a fourth aspect, there is provided a method of collecting data for determining a sphero-cylindrical refractive error of a human eye, using the apparatus of any earlier aspect, the method comprising:
positioning the first pair of apertures at the viewing position;
determining, based on the Scheiner principle, a first far point of the human eye for the first pair of first and second apertures by adjusting a distance between the human eye and a target;
positioning the second pair of apertures at the viewing position;
determining, based on the Scheiner principle, a second far point of the human eye for the second pair of first and second apertures at the second position, by adjusting a distance between the human eye and a target;
positioning the third pair of apertures at the viewing position; and
determining, based on the Scheiner principle, a third far point of the human eye for the third pair of first and second apertures at the third position, by adjusting a distance between the human eye and a target.

In accordance with a fifth aspect, there is provided a method of collecting data for determining a sphero-cylindrical refractive error of a human eye, the method comprising:
presenting the target of the second aspect to a user, at at least two different presentation angles;
providing an instruction to, or causing, the user to adjust, for each of the presentation angles, a rotational angle of:
   their eye about an axis between their eye and the target and while looking through the Scheiner component; and/or
   the current presentation angle;
   such that there is a visual indication of correspondence between the angle of the first feature and the first axis.

The visual indication may comprise the alignment of two virtual images of the first feature visible to the user.

The visual indication may comprises the overlapping of the two virtual images of the first feature visible to the user.

The method may comprise presenting the target to the user at two angles.

The method may comprise presenting the target to the user at three or more angles.

Optionally, angles at which the target is presented to the user may be equidistant.

The method may comprise presenting the target to the user at exactly three equidistant angles.

### BRIEF DESCRIPTION OF THE DRAWINGS

FIGS. 1 to 4 show front elevations of implementations of an apparatus for use in collecting data for determining a sphero-cylindrical refractive error of a human eye;

FIG. 5 is a perspective view of a laptop for implementing a method of collecting data for determining a cylindrical refractive error of a human eye;

FIG. 6 is a schematic of the laptop of FIG. 5;

FIG. 7 is a flowchart showing a method of collecting data for determining a cylindrical refractive error of a human eye;

FIG. 8 shows a clock target for use in determining a cylindrical axis of a human eye;

FIGS. 9 and 10 show front elevations of implementations of a further apparatus for use in collecting data for determining a cylindrical refractive error of a human eye;

FIGS. 11 and 12 show front elevations of implementations of a further apparatus for use in collecting data for determining a cylindrical refractive error of a human eye;

FIGS. 13-15 show what a user sees when looking through the apparatus of FIGS. 11 and 12 at different distances, with the disc at a first angle;

FIGS. 15-18 show what a user sees when looking through the apparatus of FIGS. 11 and 12 at different distances, with the disc at a second angle orthogonal to the first angle;

FIG. 19 is a front elevation of an alternative implementation of an apparatus for use in collecting data for determining a sphero-cylindrical refractive error of a human eye;

FIG. 20 is a front elevation of a component of the apparatus of FIG. 19;

FIG. 21 is a front elevation of a component of the apparatus of FIG. 19; and

FIG. 22 is a front elevation of an alternative implementation of an apparatus for use in collecting data for determining a sphero-cylindrical refractive error of a human eye;

FIGS. 23-25 are front elevations of the disc and window of the apparatus of FIGS. 19-21 at different positions;

FIG. 26 is a flowchart showing a further method of collecting data for determining a cylindrical refractive error of a human eye;

FIGS. 27-36 show implementations of targets for use with an apparatus for use in collecting data for determining a sphero-cylindrical refractive error of a human eye;

FIGS. 37-39 show the target of FIG. 32 presented at various angles;

FIGS. 40-42 show what is seen by a user observing the target of FIG. 32, at the angle of presentation shown in FIG. 37, at different angles and distances;

FIG. 43-45 show what is seen by a user observing the target of FIG. 32, at the angle of presentation shown in FIG. 38, at different angles and distances;

FIG. 46 shows an alternative implementation of a target for use with an apparatus for use in collecting data for determining a sphero-cylindrical refractive error of a human eye;

FIGS. 47-48 show what is seen by a user observing the target of FIG. 46, at different angles and distances;

FIG. 49 shows an alternative implementation of a target for use with an apparatus for use in collecting data for determining a sphero-cylindrical refractive error of a human eye; and

FIG. 50 shows a method of collecting data for determining a sphero-cylindrical refractive error of a human eye.

### DETAILED DESCRIPTION OF THE INVENTION

The invention is concerned with an apparatus for use in collecting data for determining a sphero-cylindrical refractive error of a human eye, and a related method.

The invention will now be described in more detail, without limitation, with reference to the accompanying Figures.

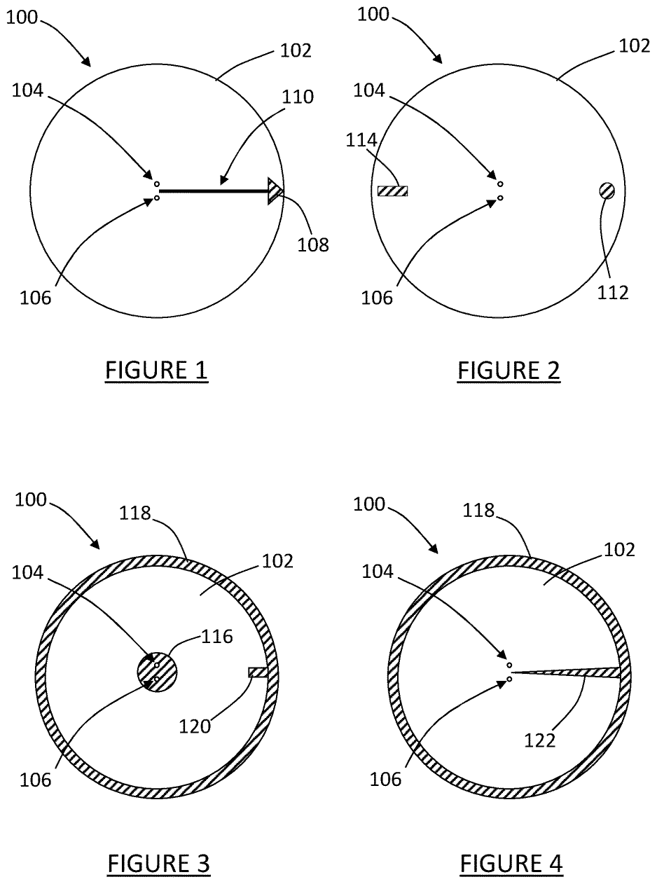

In FIG. 1, there is illustrated an apparatus 100 for use in collecting data for determining a sphero-cylindrical refractive error of a human eye. The apparatus has been developed mainly for use in collecting data that may be used to estimate an angle and power of astigmatism affecting a human eye in both cylindrical axes, thereby allowing calculation of the sphero-cylindrical refractive error, and will be described with reference to this application.

Apparatus 100 comprises a Scheiner component in the form of a flat disc 102 that is generally circular in plan. The skilled person will appreciate that other shapes may be used, but disc 102 being circular may aid a user's ability to manually rotate it when apparatus 100 is in use.

Disc 102 includes a first aperture 104 and a second aperture 106. First aperture 104 and second aperture 106 are approximately 1 mm in diameter, and their centres are spaced apart by approximately 2 mm.

The apertures can be of any suitable diameter, depending upon the implementation. For example, the apertures may have a diameter of between about 0.75 mm and 1.25 mm, or between about 0.8 mm and 1 mm, although diameters outside of those ranges may be used in certain circumstances. The apertures may be spaced apart by any suitable distance, depending upon the implementation. For example, the apertures may be spaced apart by a distance of about 1.5 mm to 4 mm, although spacings outside of that range may be used in certain circumstances. In general, a greater spacing may allow at least some users to see the two discreet images more clearly, while a lesser spacing may allow at least some users to see the two images more easily. For many users, exceeding a spacing of about 4 mm makes it difficult to see through both holes at the same time.

Disc 102 can be of any suitable thickness. It has been found that a thickness of the portion of disc 102 through which apertures 104 and 106 are formed can impact performance in certain circumstances. While a lower thickness may offer better performance, thinner materials may be less robust, harder to work with, and/or more expensive. A disc thickness of between about 0.2 mm to 2 mm has been found satisfactory, although a thickness outside of that range may be used in certain circumstances. The entire disc need not be this thick. For example, disc 102 can comprise a relatively thin portion within which apertures 104 and 106 are formed, mounted within a thicker supporting portion.

The diameters of first aperture 104 and second aperture 106 and their relative spacing means that flat disc 102 operates according to a principle (hereinafter the “Scheiner principle”) for measuring the refractive power of the eye, as observed by Scheiner in the seventeenth century. In the context of the present disclosure, the Scheiner principle involves placing a disc, such as disc 102, in front of a user's eye, and viewing a small target object through first and second apertures 104 and 106. The object will appear to be duplicated at all distances other than that at which the eye is focused.

Apparatus 100 also includes orientation indicia, in this case disposed on disc 102. The orientation indicia can include rotational indicia to allow a rotational position of the apertures relative to a user's eye to be determined when apparatus 100 is in use, as will be described in more detail below. Such orientation indicia can be printed, embossed, stamped, engraved, etched, cut, or otherwise formed on, in, or through a surface of apparatus 100.

While the indicia will typically be human-viewable, indicia that are only discernable by a particular image capture system may be used. For example, UV- or infrared-reflective ink may be used, such that the indicia are not visible to the unaided human eye, but can be captured by an image capture system capable of “seeing” the corresponding wavelengths.

In FIG. 1, the orientation indicia includes a triangular element 108 disposed near an edge of the disc 102, and a line 110 extending from the triangular element to the centre of disc 102. Together, triangular element 108 and line 110 enable an image capture and recognition system to determine an orientation of disc 102, as described in more detail below.

FIGS. 2 to 4 show other examples of orientation indicia.

FIG. 2 shows disc 102 with orientation indicia in the form of a solid circle 112 adjacent an edge of disc 102, and a solid, radially-extending rectangle 114 adjacent an opposite edge of disc 102 from circle 112.

FIG. 3 shows disc 102 with orientation indicia in the form of a solid circle 116 covering first aperture 104 and second 106, a line 118 extending around the circumference of disc 102, and a rectangle 120 extending radially inwardly from line 118.

FIG. 4 shows disc 102 with orientation indicia in the form of line 118 extending around the circumference of disc 102, and a wedge 122 extending radially from line 118 into the centre of disc 102, terminating between first aperture 104 and second aperture 106.

The skilled person will appreciate that the orientation indicia can take any other suitable form that allows for rotational orientation of disc 102 to be determined.

Apparatus 100 can be used to collect data for determining a sphero-cylindrical refractive error of a human eye. There are a number of ways in such data may be collected, examples of which will now be described.

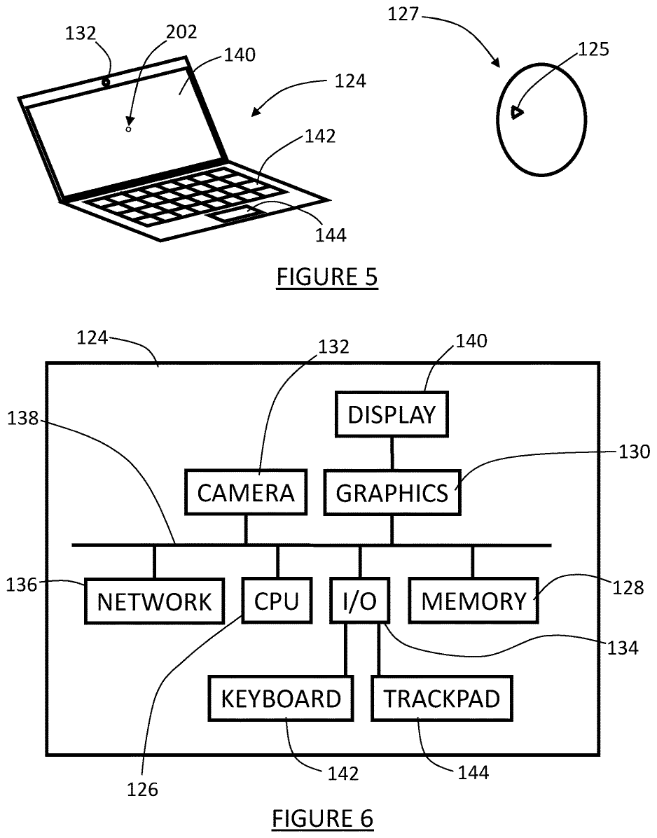

Referring to FIGS. 5 and 6, in one example, a laptop 124 is used to collect data for determining a sphero-cylindrical refractive error of an eye 125 of a human user 127. Laptop 124 includes a processor in the form of a CPU 126, memory 128 (including volatile memory such as DRAM and non-volatile memory such as a solid-state hard-drive), a graphics processor 130, an image capture device in the form of a camera 132, an I/O system 134, and a network interface 136 all connected to each other by one or more bus systems and connectors, represented generally in FIG. 5 as a bus 138.

Graphics processor 130 outputs graphics data for display on a display 140.

I/O system 134 accepts user inputs from a keyboard 142 and a trackpad 144.

Memory 128 stores software including an operating system and one or more computer software programs. CPU 128 is configured to execute the operating system and computer programs stored by memory 128. The computer program(s) stored by memory 128 include instructions for implementing any and all of the steps of the methods described in the current application.

Camera 132 captures still images and video, including still images and video of a user in certain circumstances, as described in more detail below.

Network interface 136 is configured to communicate through a network via a switch, router, wireless hub, or telecommunications network (not shown), optionally including the Internet.

The hardware of laptop 124 is conventional, and so is not described in further detail.

The skilled person will appreciate that hardware and systems for implementing the described aspects can take any suitable form, including integrated systems in which all hardware forms part of a single device such as a mobile telephone or laptop, or a distributed system, where at least some individual components form part of different devices. For example, the display can take the form of a television, mobile phone, computer tablet, or computer monitor.

Optionally, the display can be a touchscreen, which accepts user input in addition to (or instead of) keyboard 142 and trackpad 144. Any required user input can be obtained via the device that displays the images, or via any other suitable input device. For example, a mobile telephone may be used to accept user input, while images are displayed on an Internet-connected television receiving image data remotely via the Internet or a local area network, or by casting from the mobile telephone. Some or all of the computer software instructions can be stored and/or run on a remote computer such as a server.

Referring to FIG. 7, using suitable software running on laptop 124, a computer-implemented method 146 of collecting data for determining a sphero-cylindrical refractive error of a human eye may be performed.

Before moving to a description of the steps of method 146, some general principles regarding the characterization of sphero-cylindrical refractive errors of the human eye will briefly be explained.

Cylindrical refractive errors are sometimes referred to as astigmatism. Astigmatism is a rotational irregularity resulting from, for example, a barrel-shaped cornea and/or lens. This causes the eye to have different focal lengths in different radial planes through the eye.

Astigmatism can be measured and characterized by a pair of refractive power values (measured in diopters, for example) at respective orthogonal cylindrical axes of the eye. One value represents the maximum refractive power, and is associated with a first angle representing a cylindrical axis at which that maximum power occurs. The other value represents the minimum refractive power, and is associated with a second angle (offset 90° from the first angle) representing the other cylindrical axis at which that minimum power occurs.

Non-astigmatic myopes have what is called a “far point” (punctum remotem) at a distance in front of their eyes. This is a point where their uncorrected vision will be in focus, so that diverging rays from that point will be focused by the eye's optical media onto the retina. This is why myopes can see things near to them and the term “short sighted” is often used.

As known to the skilled person, the required correcting lens (i.e., the refractive error (F) can be determined by taking the reciprocal of the far point (measured in metres).

For example, if the far point is at 25 centimetres, this will lead to the calculation: F=1/−0.25=−4.00 D (Dioptres)

By adjusting a rotational position of disc 102 such that first and second apertures 104 and 106 are aligned with a first cylindrical axis of the eye, it is possible to measure a level of myopia specifically along that axis, in accordance with the Scheiner principle. Disc 102 is then rotated by 90° and a level of myopia along that axis can also be measured in accordance with the Scheiner principle. Knowing the angle and levels of myopia along these axes allows the eye's astigmatism to be characterized. The measurement of the two axes can be performed in any suitable order.

Where the user is not myopic—i.e., is hyperopic, has mixed astigmatism (myopic in one cylindrical axis and hyperopic in the other), or has low myopia (below −1.50 D, for example), artificial myopia can be introduced by way of a convex lens of known refractive power, as will be described in more detail below. That refractive power is subtracted from the measured refractive errors measured at the two axes to determine the eye's principal astigmatic cylindrical axes.

Method 146 comprises determining 148 a rotational angle of a first cylindrical axis of an eye for which data is to be collected. The skilled person will appreciate that the first cylindrical axis may correspond with either the maximum or minimum refractive power of the eye being measured but, as the skilled person will understand, it is possible to calculate and represent the sphero-cylindrical refractive error in two ways (known as the positive cylinder or the minus cylinder).

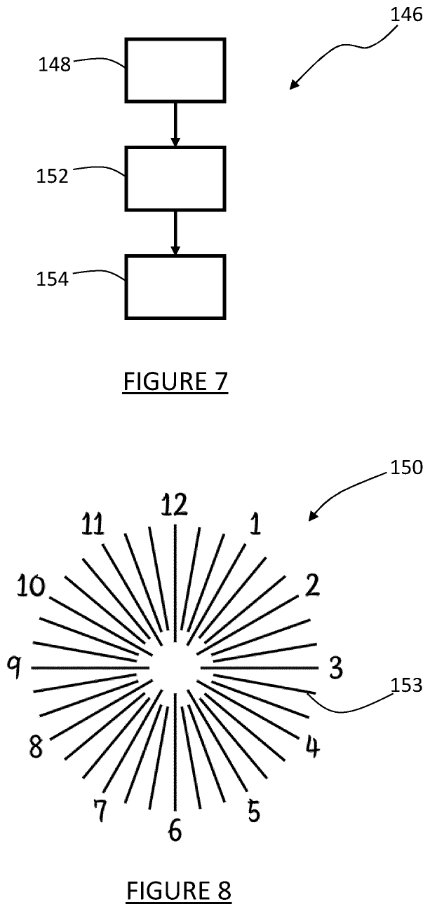

The rotational angle can be measured in any suitable way. For example, a user can be presented with a clock target 150 comprising a series of angularly spaced radial lines 153, as shown in FIG. 8. In one implementation, clock target 150 is presented on display 140 of laptop 124. The user is instructed to position themselves a suitable distance from display 140. Such instructions can be given by a technician implementing the test, or can be presented by way of written instructions on display 140, audible instructions played by laptop 124, an animation played on the laptop, any combination of these presentation options, or in any other suitable way. All subsequent instructions to the user may be given in similar way(s).

The user is instructed to indicate the numbers corresponding to the lines that appear clearest on clock target 150. This indication can be made verbally, for example to a technician implementing the test, or can be by way of interaction with laptop 124, for example by clicking at a suitable point on the clock, selecting from the menu, typing an answer, speaking into a microphone of laptop 124, any combination of these indication options, or in any other suitable way. All subsequent inputting of data or information may be done in similar way(s).

Software running on laptop 124 can accept the numbers as input data. Alternatively, instead of inputting the numbers corresponding to lines that appear clearest on clock target 150, the user can be instructed to use that information in the next step of method 146.

Determining 148 the rotational angle of the first cylindrical axis of the eye for which data is to be collected may also include identifying, looking up, or otherwise referencing such a rotational angle that has previously been determined by other means. For example, determining 148 the rotational angle may include looking up the rotational angle in a memory, such as in a database. The memory can be memory 128 of laptop 124, or can be remote memory, such as in a server accessible via a local network and/or the Internet.

Returning to method 146, a first far point of the user's eye is determined 152 using apparatus 100.

The user can be instructed to orientate disc 102 based on the determined rotational angle. If the rotational angle was measured, for example by way of the user's interaction with clock target 150, that information can be used to determine an appropriate rotational position of disc 102 during the step of determining 152 the first far point.

Optionally, the orientation indicia can include information corresponding at least in part with the information displayed on clock target 150. For example, some or all of the numbers shown on clock target 150 can be replicated on disc 102, positioned at a suitable angular position relative to first and second apertures 104 and 106. This allows the rotational angle determined by the user's interaction with clock target 150 to be used directly to rotationally position disc 102 as part of step 152. For example, if the user interacts with clock target 150 and determines that the radial line nearest the number “4” is clearest, then the user may be instructed to rotate disc 102 such that the number “4” on disc 102 points directly upwards.

Where no such corresponding indicia are provided on disc 102, the user may be directed to move a particular element of the indicia that is provided on disc 102 to a particular orientation. For disc 102 of FIG. 1, for example, the user may be instructed to position disc 102 in front of their eye with the triangular element 108 pointing at the same angle as the “4” in clock target 150. Alternatively, if the indicia includes letters or shapes spaced around the edge of disc 102, the user may be instructed to position disc 102 such that a particular letter or shape points directly upwards.

Once the disc is at the correct rotational position, it is brought close to the eye so that the first far point can be determined based on Scheiner principles.

Determining the first far point based on Scheiner principles requires the user to move relative to a target point. The target point can be, for example, a dark circle on a light background, or vice versa. Alternatively, the target point can take the form of a recognizable image, which will generally be compact in form for best outcomes. The image can be displayed on laptop 124 for example, or can take the form of a physical item (such as a printed card) placed on a wall or other vertical surface. Alternatively, the target point can be displayed on a television, tablet, or other display.

The target point is initially viewed at a suitable distance. For example, the user may be asked to stand at least a predetermined distance from display 140, if the target point is presented on laptop 124, or from the physical item.

The user places disc 102 close to their eye, at the rotational angle determined in step 148 and with first and second apertures 104 and 106 between the target point and their eye. At this time, the user should see two copies of the target point.

The user is instructed, for example by a technician or software running on laptop 124, to slowly move closer to the target point until it resolves into a single target point. The distance between the eye and the target point corresponds to the eye's far point at that particular rotational angle.

The distance can be determined in any suitable way. For example, the user (or a technician) may use a measuring device, such as a rule, tape measure, or laser measure, to determine the distance. Camera 132 of laptop 124 can capture an image of the user, and the distance can be estimated based at least in part on processing of the image. For example, the captured image can be processed by software running on laptop 124 (or remotely) to estimate the distance based on a spacing between the eyes, nose, and or mouth of the user.

Alternatively, or in addition, indicia on apparatus 100 in the captured image can be used to estimate the distance. The orientation indicia can be used for this purpose. Alternatively, or in addition, further indicia that may be better suited to size (and therefore distance) estimation can be included on apparatus 100. For example, a high-contrast scale (not shown) may be printed onto a surface of disc 102 for capture in an image, thereby allowing the distance to be estimated.

The estimated distance is recorded. For example, the distance can be manually input into laptop by way of keyboard 142 or trackpad 144, for example. Alternatively, if laptop 124 is configured to estimate the distance using camera 132 as described above, the result can be recorded automatically, optionally without any user interaction.

Next, a second far point of the user's eye is determined 154 using apparatus 100, with disc 102 orientated orthogonally to an angle at which the first far point was determined.

Optionally, the user can be instructed to orientate disc 102 in this manner, based on the determined rotational angle. If the rotational angle was measured, for example by way of the user's interaction with clock target 150, that information can be used to determine a rotational position of disc 102 during the step of determining 154 the second far point.

If the orientation indicia includes information corresponding at least in part with the information displayed on clock target 150 as described above, the rotational angle determined by the user's interaction with clock target 150 can be used directly to rotationally position disc 102 as part of step 152. For example, if the user interacts with clock target 150 and determines that the radial line nearest the number “4” is clearest, then the user may be instructed to rotate disc 102 such that a particular element of the rotational indicia is at a rotational position that is 90° offset from the rotational position of the number “4” on clock target 150. For disc 102 of FIG. 1, for example, the user may be instructed to position disc 102 in front of their eye with the triangular element 108 pointing at an angle corresponding to as the “1” in clock target 150 (the “1” being 90° offset from the “4”).

Once the disc is at the correct rotational position, it is brought close to the eye so that the second far point can be determined based on Scheiner principles, in a similar manner as was described in relation to the first far point. The user places disc 102 close to their eye, at a rotational angle 90° offset from the rotational angle determined in step 152 and with first and second apertures 104 and 106 between the target point and their eye. At this time, the user should see two copies of the target point.

The user is then instructed, for example by a technician or software running on laptop 124, to slowly move closer to the target point until it resolves into a single target point. The distance between the eye and the target point corresponds to the eye's far point at that particular rotational angle.

The distance can be determined in any suitable way, such as in any of the ways described above.

The estimated distance is recorded, optionally in the same manner as was done for the distance estimated when determining the first far point.

Based on the determined angle and the recorded distances, software can be used to characterize both astigmatic focal powers and thus the sphero-cylindrical refractive error of the user's eye. The software can be run on laptop 124, or information relating to the angle and recorded distances can be forwarded to a server via a local network and/or the Internet for remote processing.

Method 146 can then be repeated for the user's other eye.

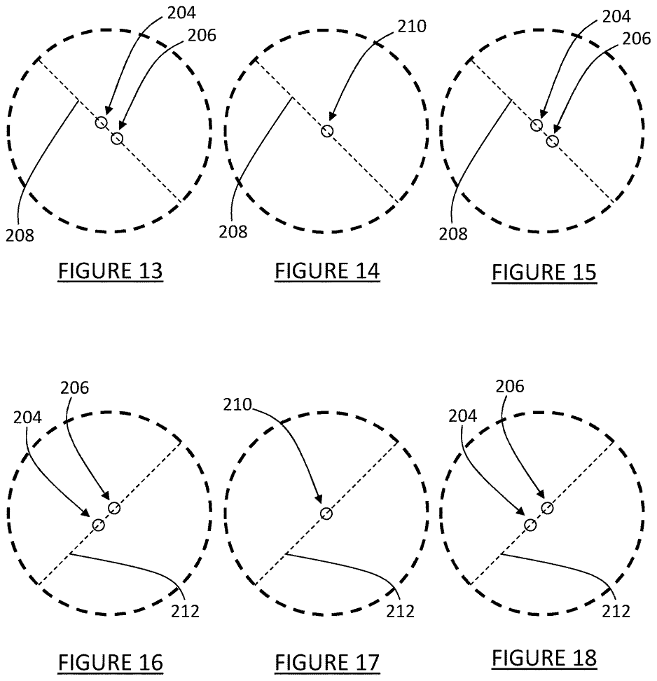

Turning to FIG. 13-18, there is shown a sequence of schematic views of what a user sees when using implementations of the apparatus. The illustrated view in each example is a small subset of the visual field. The schematic views are based on the presentation of a white dot 202 against a dark background on display 140 of laptop 124. For clarity, the dark background in FIG. 5 is shown without shading.

The skilled person will appreciate that other colours and shades can be used. For example, a dark dot on a light background, or a dot of a colour, shade, pattern, or appearance that is of relatively high contrast to a different background colour, shade, pattern or appearance.

It will be appreciated that dot 202 may alternatively be displayed on another display, such as a mobile phone or tablet display, or a television screen, for example. While there may be advantages, in particular implementations, involved with presenting dot 202 on an emissive display such as these, in other implementations, dot 202 may be present on a reflective surface. For example, dot 202 can be printed, embossed, engraved, or otherwise marked onto a background surface.

The skilled person will also appreciate that an image other than a dot may be used in other implementations. For example, a small image of an object, such as a car or a balloon, can be presented against a contrasting background.

Optionally, the background may include a pattern or other imagery. Such imagery can optionally include imagery that encourages the user to relax the focus of the eye being tested, to minimize the effect of accommodation. For example, the imagery can include a distant mountain with a road appearing to recede from the user and up the mountain. The dot or other image can be positioned some way up the road, which subliminally encourages relaxation of the eye's focus.

In one implementation, dot 202 is about 5 mm in diameter, which when viewed from a distance of between about 200 and 500 mm subtends an arc of about 0.5° to 1.0°. This corresponds to about 1/10 of the fovea. However, the skilled person will appreciate that dot 202 (or other image) can be larger or smaller, and can be sized to cover different proportions of the fovea.

Because the projection of images onto the retina (as described in more detail below) is an illusion created by the apertures and the optics of the eye, neither the diameter of the pinholes themselves, nor the size of the display is strictly relevant to the visual field needed on the retina to see the two spots. However, at least in one implementation, the aperture spacing and range of intended viewing distances are selected to keep the virtual images within about 7° of each other at maximum separation, which can provide good visibility of the virtual images and their spacing for most users.

Returning to FIGS. 13-18, once the first cylindrical axis has been determined as described above, and disc 102 rotated to a corresponding angular position, the apparatus is positioned in front of the eye to be tested, with the user at a known distance to dot 202. This initial distance can be determined in any suitable way, including in the various ways described above, but need not be known to a high degree of accuracy at this stage.

Initially, the user will see a first virtual dot 204 and a second virtual dot 206, as shown in FIG. 13. Virtual dots 204 and 206 are aligned as indicated by line 208.

The user moves closer to dot 202. As the user approaches the first far point, virtual dots 204 and 206 appear to converge into a single dot 210, as shown in FIG. 14. The distance to dot 202 at this position represents the first far point described above.

If the user moves beyond the first far point (i.e., closer to display 140), virtual dots 204 and 206 diverge, as shown in FIG. 15.

The user can optionally move backwards and forwards until they reach the point of maximum convergence as shown in FIG. 14.

The first far point can then be determined by measuring the distance to the dot 202 when the virtual dots are converged. That distance can be determined in any suitable way, including in the various ways described above.

Next, disc 102 is rotated 90°, and the process is repeated. The apparatus is positioned in front of the same eye, with the user at a known distance to dot 202. The user will again see first virtual dot 204 and second virtual dot 206, as shown in FIG. 16. However, this time virtual dots 204 and 206 are aligned as indicated by line 212.

The user moves closer to dot 202. As the user approaches the second far point, virtual dots 204 and 206 appear to converge into a single dot 210, as shown in FIG. 17. The distance to dot 202 at this position represents the second far point described above.

If the user moves beyond the second far point (i.e., closer to display 140), virtual dots 204 and 206 diverge, as shown in FIG. 18.

The user can optionally move backwards and forwards until they reach the point of maximum convergence as shown in FIG. 17.

The second far point can then be determined by measuring the distance to the dot 202 when the virtual dots are converged. That distance can be determined in any suitable way, including in the various ways described above.

The distances corresponding to the first and second far points can then be recorded manually, or automatically if they have been determined automatically in one of the ways described above.

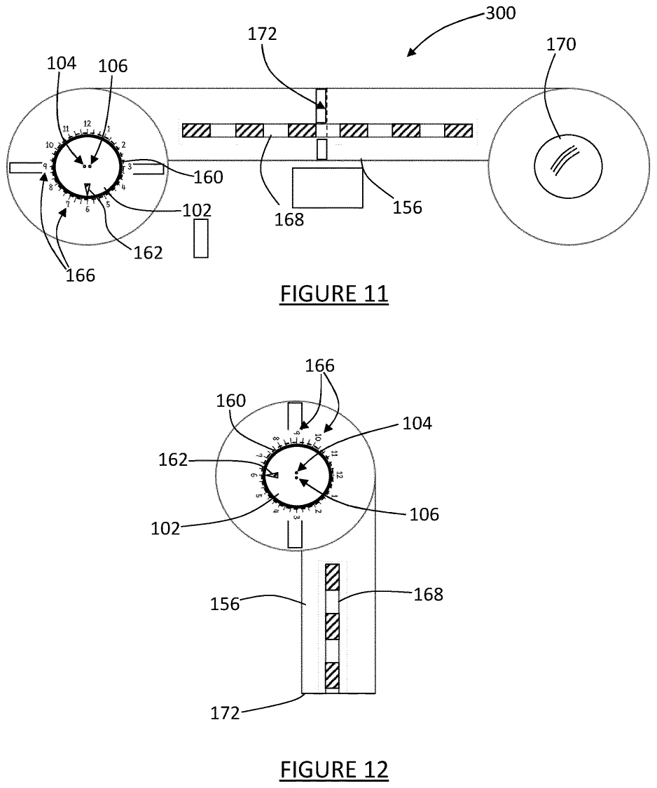

As explained above, where the user is not myopic—i.e., is hyperopic, has mixed astigmatism (myopic in one cylindrical axis and hyperopic in the other), or has low myopia (below −1.50 D, for example), artificial myopia can be introduced by way of a convex lens of known refractive power. For example, a separate convex lens of known refractive power (+8.0 D, for example) can be provided for attachment to apparatus 100. The use of such a convex lens will be described in more detail below in relation to the implementation of FIGS. 11 and 12.

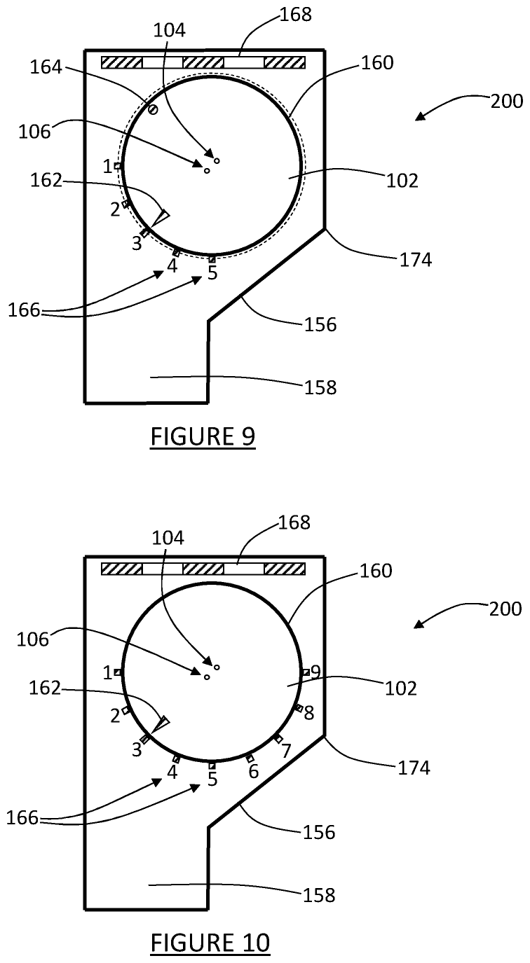

FIG. 9 shows a further implementation of an apparatus 200 for use in collecting data for determining a sphero-cylindrical refractive error of a human eye. Apparatus 200 shares several elements with apparatus 100, and such elements are indicated by the same reference numbers.

In apparatus 200, the Scheiner component in the form of disc 102 is mounted for rotation relative to a frame 156. Frame 156 be formed from any suitable material, such as cardboard or a polymeric material.

Frame 156 includes an optional handle 158 that can be grasped by a user when apparatus 200 is in use. Disc 102 is mounted for rotation by way of a groove 160 formed in frame 156 that supports an outer edge of disc 102. Disc 102 can be rotated such that its edge slides relative to groove 160.

The orientation indicia of apparatus 200 includes indicia on the Scheiner component (i.e., disc 102) in the form of a radially outwardly pointing arrow 162 and a dot 164, both positioned near the edge of disc 102.

Apparatus 200 also includes frame indicia in the form of angle indicators 166 on frame 156. Angle indicators 166 are numbered 1 to 5 in this implementation, the numbers respectively corresponding to 90°, 67.5°, 45°, 22.5°, and 0° from vertical, but any other form of indicia including letters, symbols, or the like, can be used in other implementations. The resolution can be higher or lower than the 22.5° resolution of the illustrated implementation. Similarly, although arrow 162 and a dot 164 are used on disc 102, any other suitable indicia can be used in other implementations.

Apparatus 200 can be used to implement method 146, with some slight differences to account for the differences between apparatus 100 and apparatus 200. The rotational angle is determined, for example as described above in relation to apparatus 200. The user is then instructed to rotate disc 102 relative to frame 156 until arrow 162 is aligned with a particular number. For example, if the first cylindrical axis is determined to be at 45° to the vertical, the user can be instructed to rotate disc 102 until arrow 162 is aligned with the number 3, as shown in FIG. 9. As above, the instructions can be given by a technician, or via software running on laptop 124. The user then determines the first far point, for example as described above.

The user is then instructed to rotate disc 102 until dot 164 is aligned with the number 3. Since dot 164 is offset from arrow 162 by 90°, the effect of this is to rotate the axis through first aperture 104 and second aperture 106 by 90°.

The user then determines the second far point, for example as described above. The use of indicia on both disc 102 and frame 156 may make it easier for a user to accurately position disc 102 at particular angles when determining the first and second far points.

The frame indicia on frame 156 includes an optional scale 168, which can be used in determining distance as part of the first and second far points, and/or determining whether the user has aligned apparatus 200 correctly. Scale 168 includes alternating black and white segments. When using camera 132 (or another camera) to determine a distance of apparatus 200 from the target point, scale 168 offers a high-contrast target image of known size, which may increase the accuracy and/or reliability of image processing to determine the distance.

The skilled person will appreciate that other types of scale may be user, including one or more lines, grids, dots, shapes, or any other feature allowing for image processing software to estimate the scale's relative size, and hence determine distance.

Although apparatus 200 shows angle indicators 166 on frame 156, and arrow 162 and dot 164 on disc 102, the skilled person will appreciate that the positions of these elements may be swapped. That is, angle indicators 166 may be disposed on disc 102, while arrow 162 and dot 164 may be disposed on frame 156. The principle of aligning arrow 162 and then dot 164 with a particular angle indicator 166 still applies. As with the implementation of FIG. 9, the angle indicators and other indicia can take any other suitable form.

Instead of having two indicators (i.e., arrow 162 and dot 164) offset by 90°, a single such indicator can be used. In that case, angle indicators can be extended to subtend 180° rather than the 90° as shown in FIG. 9. For example, as shown in FIG. 10, angle indicators 166 include numbers 1-5 as shown in FIG. 9, but also numbers 6-9. Only arrow 162 is disposed on disc 102 (i.e., dot 164 is omitted). The user is instructed to rotate disc 102 until arrow 162 is aligned with one of the numbers 1-9 for determining the first far point, The user is then instructed to rotate the disc to a second one of the numbers 1-9, which is 90° offset from the first number, for determining the second far point.

The skilled person will appreciate that other arrangements and types of angle indicators can be used, optionally extending up to 360° around disc 102.

Frame 156 includes a bridge-engaging portion 174, which is an edge of frame 156 intended to engage a bridge of a user's nose when apparatus 200 is in use. This stabilizes apparatus 200 relative to the user's face.

As with apparatus 100, apparatus 200 can optionally include a positive power lens (not shown), such as a convex lens, for generating a positive refractive offset. Such a lens can be mounted in front of or behind first and second apertures 104 and 106. The use of such a lens will be described in more detail below, with reference to FIGS. 11 and 12.

FIGS. 11 and 12 show a further implementation of an apparatus 300 for use in collecting data for determining a sphero-cylindrical refractive error of a human eye. Apparatus 300 shares several elements with apparatus 200, and such elements are indicated by the same reference numbers.

In FIGS. 11 and 12, angle indicators 166 on frame 156 are numbered 1-12 adjacent to the periphery of disc 102. The numbers 1-12 are positioned to correspond with the same numbers in clock target 150. In this way, the user's interaction with clock target 150 can be mapped directly to adjusting the position of disc 102 (and arrow 162, in particular) relative to angle indicators 166. The angle indicators can alternatively be positioned on disc 102, with arrow 162 (or other indicator) being positioned on frame 156 adjacent to disc 102.

In other implementations, angle indicators 166 do not relate to clock target 150 or anything else used to initially determine a cylindrical axis as described earlier, and are instead used as an index for the angular position of disc 102 relative to frame 156.

Apparatus 300 comprises a positive power (i.e., convex) lens 170 mounted to frame 156. Lens 170 is of a known power (e.g., +8.0 D). Frame 156 has a fold line 172 about which frame 156 can be folded.

When not folded about fold line 172, as shown in FIG. 11, lens 170 is positioned away from first and second apertures 104 and 106.

When it is desired to use lens 170, frame 156 is folded about fold line 172, as shown in FIG. 12. This places lens 172 behind first and second apertures 104 and 106 (i.e., closer to the user's eye).

Method 146 can be performed, for example as described above, with lens 170 in front of first and second apertures 104 and 106. When determining the refractive error implied by the first and second far points, a correction factor based on the power of lens 170 is applied. For example, if lens 170 is a +8.0 D lens, then 8.0 D is subtracted by the software that is calculating the refractive errors required to characterize the user's astigmatic cylindrical axes and thence sphero-cylindrical refractive error.

Whether to use lens 170 may be determined in any suitable manner. For example, if the user is known to have myopia, for example as a result of previous testing, it may be known that it is not necessary to use lens 170. If the user is known to be hyperopic, emmetropic, or insufficiently myopic, they may be instructed to use lens 170. Alternatively, a preliminary questionnaire and/or eye tests may be used to coarsely estimate whether the user is likely to require use of lens 170.

Lens 170 may alternatively be mounted to an apparatus such as apparatus 100, 200, or 300 in a removable way. For example, the apparatus may include a lens mount for mounting the lens. The lens mount can take the form of, for example, a groove or shelf on disc 102 or frame 156 into/onto which lens 170 sits.

The skilled person will appreciate that there are many other potential ways in which lens 170 may be mounted to an apparatus, such as apparatus 100, 200, or 300, whether permanently or temporarily.

When a lens, such as lens 170, is available, method 146 may optionally include determining whether a refractive error of an eye for which data is to be collected is greater than, or likely to be greater than, a predetermined value. For example, the user can complete a questionnaire about their eyesight, which may provide an indication that the refractive error of their eyes is greater than, or likely to be greater than, a threshold. Alternatively, or in addition, the user's age may be taken into account when determining a likelihood of the refractive error of their eyes being greater than a threshold. Alternatively, or in addition, the user may undertake a basic eye test to give at least a coarse indication of whether the refractive error of their eyes is greater than a threshold. Such an eye test need only be rudimentary, such as asking the user to determine whether they can read characters of known sizes at a known distance (e.g., at arm's length).

An example of such a threshold is −1.50 D. That is, it is determined whether each of the user's eyes is (or is likely to be) hyperopic, emmetropic, or slightly myopic (i.e., less than −1.50 D in this example). The skilled person will appreciate that any suitable threshold may be used, although in general the threshold will tend to lie withing the low to medium myopia range.

Responsive to determining that the refractive error of the eye is greater than the predetermined value, the lens is positioned relative to the apparatus such that it is in front of a user's eye when the apparatus is in use, prior to determining the first and second far points.

Positioning the positive power lens can comprise installing the lens on a lens mount of the apparatus.

Alternatively, positioning the positive power lens can comprise moving the positive power lens from a second position in which the lens is not in front of the user's eye when the apparatus is in use, to a first position.

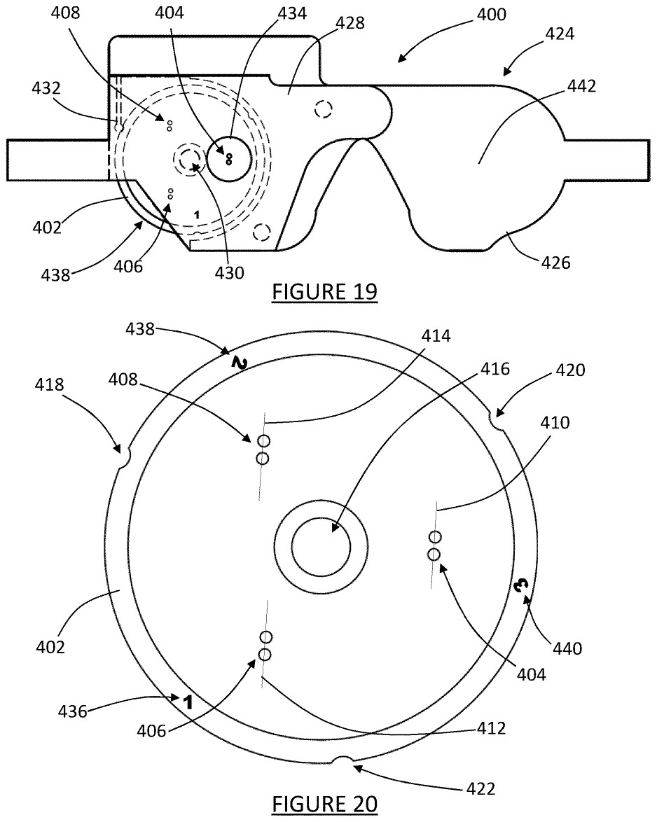

Turning to FIGS. 19 to 21 and 23, there is shown an alternative apparatus 400 for use in collecting data for determining a myopic or hyperopic sphero-cylindrical refractive error of a human eye including cylinder axis and near addition. Apparatus 400 shares several elements with apparatus 100, apparatus 200, and apparatus 300, and such elements are indicated by the same reference numbers.

In apparatus 400, the Scheiner component takes the form of a flat disc 402 that is generally circular in plan. The skilled person will appreciate that other shapes may be used, but disc 402 being circular may aid a user's ability to manually rotate it when apparatus 400 is in use.

As best shown in FIG. 20, disc 402 includes a first pair 404 of apertures, a second pair 406 of apertures, and a third pair 408 of apertures. Each of the first, second, and third pairs 404, 406 and 408 of apertures comprises first and second apertures having similar size and spacing to first aperture 104 and second aperture 106 described in earlier examples.

In the example illustrated in FIGS. 19-21, first, second, and third pairs 404, 406 and 408 of apertures are equally angularly spaced around the surface of disc 402. The pairs of apertures are arranged such that they are aligned along parallel axes 410, 412, and 414 (each axis 410, 412, and 414 passes through one of the pairs 404, 406, and 408 of apertures), although in other implementations this need not be the case.

Disc 402 includes a central hole 416 and first, second, and third detents 418, 420, and 422 spaced equally about the periphery of disc 402.

Disc 402 includes indicia in the form of first, second, and third reference symbols 436, 438, and 440 printed near the periphery of disc 402. In the illustrated embodiment, first, second, and third symbols are the numbers ‘1’, ‘2’, and ‘3’, which have the advantage of being ordinal and familiar across many languages. In this context, “ordinal” means have a natural order, which means a user knows which way to rotate disc 402 (as described in more detail below) when apparatus 400 is in use. However, the indicia need not be ordinal, and can even take the form of shapes or symbols.

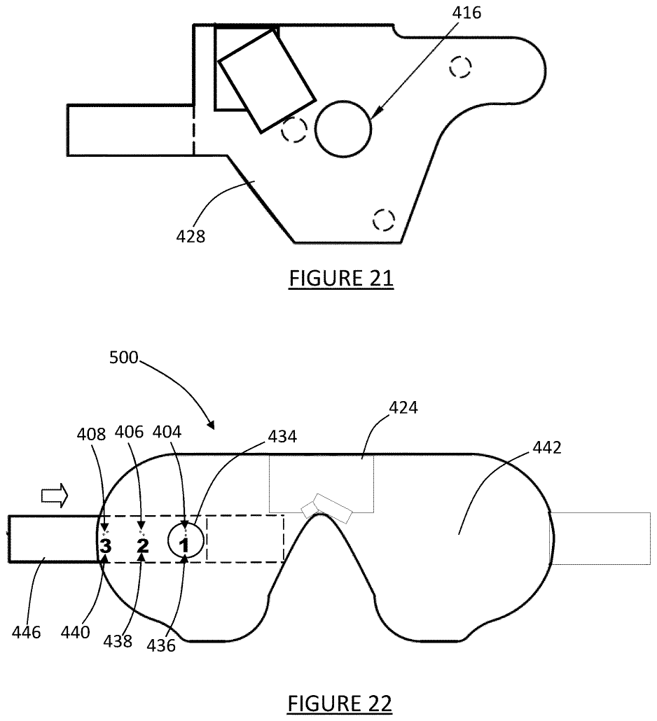

As best shown in FIG. 19, apparatus 400 includes a frame 424. Frame 424 includes a first element 426 and a second element 428 (second element 428 is shown in FIG. 21). First element 426 includes a blanking portion 442 that covers the eye not being tested.

An axle 430 is disposed between first element 426 and second element 428. Disc 402 is sandwiched between first element 426 and second element 428 such that axle 430 passes through hole 416 in disc 402. This allows rotation of disc 402 about axle 430.

Second element 428 includes a window 434 that defines a viewing position. An edge of disc 402 extends past an edge of frame 424 such that a portion 442 of the surface of disc 402 is visible. The visible portion 442 changes as disc 402 rotates.

A flexible pawl 432 is mounted between first element 126 and second element 428 such that it engages the periphery of disc 402. As the disc 102 is rotated about axle 430, pawl 432 sequentially engages each of first, second, and third detents 418, 420, 422, such that disc 402 is accurately stopped at each of three rotational positions.

Disc 402, first element 426, and second element 428 are stamped cardboard elements that are bonded together, although any other suitable material and/or manufacturing process may be used.

A means (not shown) of holding the frame against the user's face may also be provided, such as spectacle arms, an elastic or hook and loop strap, or a handle protruding from the device.

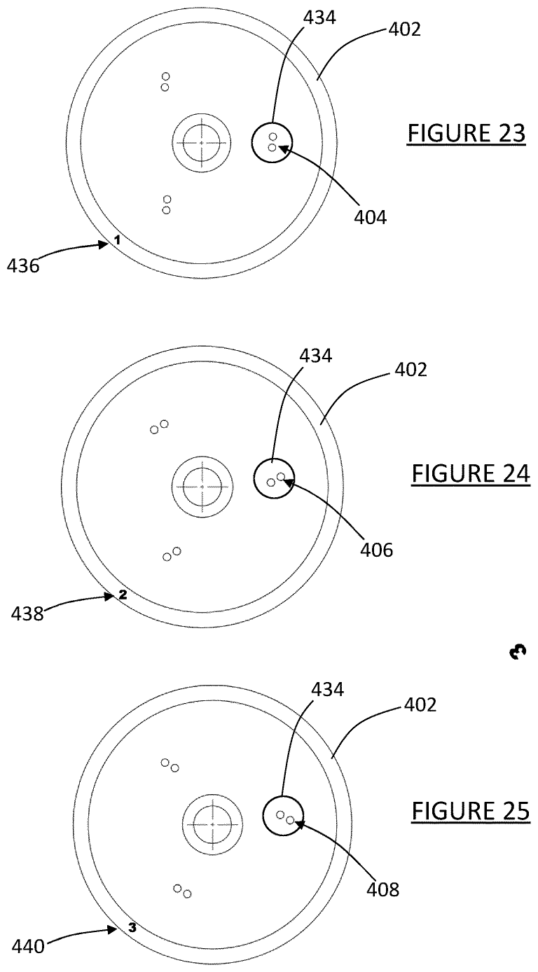

In use, disc 402 is rotated such that first symbol 436 (i.e., the number ‘1’) is visible on portion 442 of disc 402. At this point, pawl 432 engages corresponding first detent 418, which accurately positions first pair 404 of apertures within window 434. The orientation of the first pair 404 of apertures relative to window 434 when disc 402 is at this position is shown in FIG. 23 (with only first symbol 436 shown, for clarity).

Apparatus 400 is positioned in front of the user's face such that window 434 is in front of one eye. The user is instructed to view a dot (such as dot 202 or other target image as described above) through the first pair 404 of apertures from further than a predetermined distance, which can be determined in a similar manner to that described above. At the predetermined distance, the user will see a pair of dots. The other eye is covered by blanking portion 442.

The user moves towards dot 202 until the observed pair of dots converges into a single dot. At the point of convergence, the distance between first apertures 404 and dot 202 represents the far point for the eye being tested, at the angle of axis 410. The distance is determined in any suitable manner, including any of the ways described above.

Additional indicia (not shown), such as scale 168 described above, can be added to apparatus 400 to assist in the automated determination of distance.

Disc 402 is rotated until second symbol 438 (i.e., the number ‘2’) is visible on portion 442 of disc 402. At this point, pawl 432 engages corresponding second detent 420, which accurately positions second pair 406 of apertures within window 434. The orientation of the second pair 406 of apertures relative to window 434 when disc 402 is at this position is shown in FIG. 24 (with only second symbol 438 shown, for clarity).

Apparatus 400 is again positioned in front of the user's face such that window 434 is in front of the same eye. The user is instructed to view dot 202 through second pair 404 of apertures from at least the predetermined distance. At the predetermined distance, the user will again see a pair of dots.

The user moves towards dot 202 until the observed pair of dots converges into a single dot. At the point of convergence, the distance between second apertures 406 and dot 202 represents the far point for the eye being tested, at the angle of axis 412. The distance is determined in any suitable manner, including any of the ways described above.

The process is repeated for the third pair 406 of apertures. The orientation of the third pair 408 of apertures relative to window 434 when disc 402 is at this position is shown in FIG. 25 (with only third symbol 440 shown, for clarity).

With the three distances corresponding to the three positions of disc 402, a myopic or hyperopic sphero-cylindrical refractive error of a human eye can be determined in any suitable manner. One such example will now be described.

The two principal meridians with the highest difference in power are perpendicular, and the change in power over the pupil meridians follows a sine squared function. In conjunction with the determined distances for the far-point at each of the three rotational angles, this information can be used to determine the spherical power, the cylindrical astigmatism and the axis of the astigmatism.

For example, where the specific meridians used are 0°, 60°, and 120°, the sphere, cylinder and axes of the test eye may be solved from the following equation derived from the application of Euler's Law as taught by Gekeler, F., et al, (1997), “Measurement of astigmatism by automated infrared photoretinoscopy”, Optometry and Vision Science, 74(7), 472-482:

$$M = \frac{R ( 0 ) + R ( 60 ) + R ( 120 )}{3}$$

$${J}_{0} = \frac{( 2 * R ( 0 ) ) - R ( 60 ) - R ( 120 )}{3}$$

$${J}_{45} = \frac{R ( 60 ) - R ( 120 )}{\sqrt{3}}$$

$$Sphere = M + \sqrt{{J}_{0}^{2} + {J}_{45}^{2}}$$

$$Cylinder = M - \sqrt{{J}_{0}^{2} + {J}_{45}^{2}}$$

where:
   R(0), R(60) and R(120) are the powers of refraction of the eye at the meridians indicated (i.e., 0, 60° and) 120°;
   M is the average spherical equivalent of the eye;
   J₄₅ is the oblique astigmatism component; and
   J₀ is the straight astigmatism component.

Solving the Cylinder in this way gives the full power of the Cylinder. The Cylinder can also be solved as:

$$Cylinder = - 2 * \sqrt{{J}_{0}^{2} + {J}_{45}^{2}}$$

which gives the difference between the Sphere and the full power of the Cylinder as would typically be presented on an optometric prescription.

The skilled person will appreciate that additional arithmetical manipulations may be required to output a prescription in a particular standardised format.

To test the other eye, the device can be turned around such that the window is positioned in front of the other eye, and the test repeated. Depending upon the implementation and indicia, the software (or manual calculations) used to convert the measurements into a refractive error may need to take into account the fact that the disc is rotating in a different direction as a result of the frame being flipped for use with the other eye.

The skilled person will appreciate that a similar set of tests can be performed starting with the user closer than a predetermined distance to the dot (or target image). The user then moves away from the dot until it resolves from a pair of dots into a single dot. The distance should be the same, irrespective of whether the user starts at a distance and moves closer, or vice versa.

The skilled person will appreciate that the Scheiner component may take other forms. For example, the Scheiner component can take the form of a linear strip that can be translated by sliding. For example, FIG. 22 shows a further implementation of an apparatus 500 for use in collecting data for determining a sphero-cylindrical refractive error of a human eye. Apparatus 500 shares several elements with apparatus 400, and such elements are indicated by the same reference numbers.

In apparatus 500, the Scheiner component can take the form of a strip 446 that is mounted for horizontal sliding between each of three positions. At each of the positions, a different one of first, second, and third apertures 404, 406, and 408 is visible within window 434. Each pair of apertures 404, 406, and 408 is at a different rotational position on strip 446 compared to the others pairs of apertures. For example, one pair can be at 0° to the vertical, another pair at 60° to the vertical, and the other at 120° to the vertical.

Apparatus 500 is used in a similar way to apparatus 400. However, instead of rotating disc 402, strip 446 is slid relative to frame 424. The same measurements are taken, and the refractive error determined in the same way as described for apparatus 400. A detent and pawl arrangement can optionally be provided, although the fixed angle of the aperture pairs relative to the line along which strip 446 slides means accurate positioning may be of less importance.

For testing the other eye, apparatus 500 can be flipped as was described for apparatus 400. Alternatively, strip 446 can be mounted such that it can slide all the way to the other side of frame 424. In this way, the same pairs of apertures can be used for both eyes.

In yet another alternative, another strip similar to strip 446 can be mounted on the other side of the frame. Optionally, each strip can be positionable such that it covers its corresponding window while apertures of the other strip are used to measure the far points of the corresponding eye.

The skilled person will appreciate that more than three pairs of apertures can be provided. For example, four, five, or more pairs of apertures can be provided, and the far-points measured for all the pairs of apertures used as inputs to a sine-squared function similar to that described above (modified to account for the greater number of inputs). This applies to both rotational and translation Scheiner components.

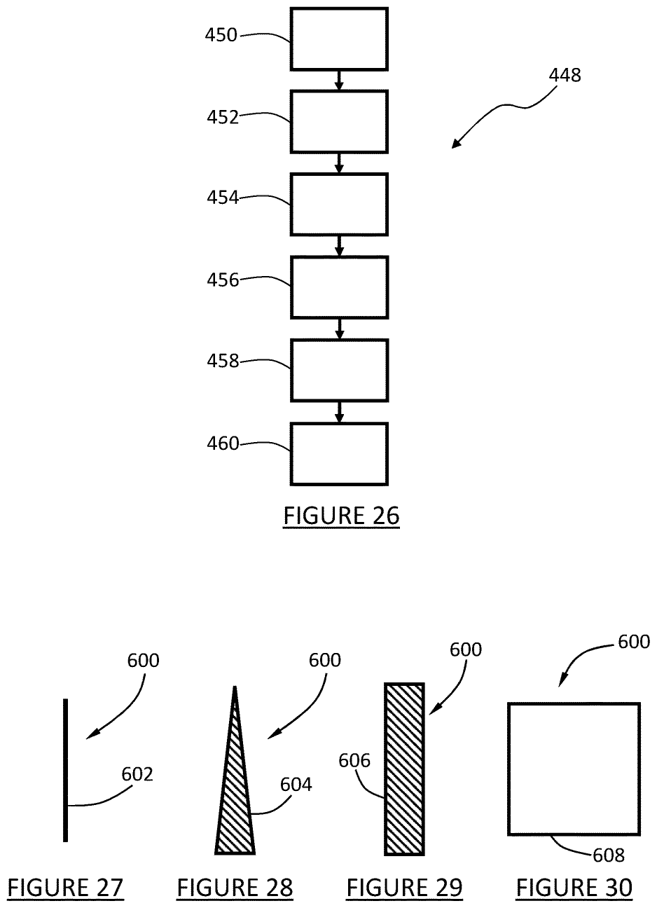

Referring to FIG. 26, there is shown a method 448 of collecting data for determining a sphero-cylindrical refractive error of a human eye. Method 448 comprises positioning 450 a first pair of apertures at the viewing position, and determining 452, based on the Scheiner principle, a first far point of the human eye for the first pair of first and second apertures by adjusting a distance between the human eye and a target. Method 448 comprises positioning 454 the second pair of apertures at the viewing position, and determining 456, based on the Scheiner principle, a second far point of the human eye for the second pair of first and second apertures at the second position, by adjusting a distance between the human eye and a target. Method 448 comprises positioning 458 the third pair of apertures at the viewing position, and determining 460, based on the Scheiner principle, a third far point of the human eye for the third pair of first and second apertures at the third position, by adjusting a distance between the human eye and a target.

The skilled person will appreciate that the steps of the method can be performed in any suitable order.

Method 448 can be used with any of apparatus 100, apparatus 200, apparatus 300, apparatus 400, apparatus 500, and any other suitable apparatus that allows the measurement of the far points of a human eye for at least three different angles.

The three angles at which aperture pairs are measured for method 448 can be provided in any other manner. For example, each pair of apertures can be disposed on a flap, and each flap can be folded into the viewing position to place its apertures at the appropriate angle. The two orthogonal angles at which aperture pairs are measured for method 146 can be provided in any similar manner.

Although the three angles at which aperture pairs are measured for method 448 have been described as being equally spaced, the skilled person will appreciate that this need not be the case. Using, for example, the sine squared equations above, any three (or more) arbitrary angles can be used. However, equally angularly spacing the aperture pairs potentially provides increased accuracy and resilience to slight measurement errors or inaccuracies in use (especially if only three angles are used).

Similarly, a Scheiner component such as disc 102, disc 402, or a similar disc, or any other shape of Scheiner component not mounted to a frame for movement, can be used freehand with suitable instructions and indicia. While the result may be less accurate depending on user and/or operator skill, the apparatus itself is greatly simplified, making it even cheaper to produce.

The skilled person will appreciate that method 448 allows for direct determination of refractive errors without the need to initially establish a meridian of maximum or minimum focal length (e.g., by way of clock target 150).

Similarly, apparatus 400 and apparatus 500 can be modified for use with method 146. In that case, only two pairs of apertures are required, having axes at 90° to each other.

As explained above, where the user is not myopic—i.e., is hyperopic, has mixed astigmatism (myopic in one cylindrical axis and hyperopic in the other), or has low myopia (below −1.50 D, for example), artificial myopia can be introduced by way of a convex lens of known refractive power. For example, a separate convex lens of known refractive power (+8.0 D, for example) can be provided for attachment to apparatus 400 or 500.

Referring to FIGS. 27 to 50, there are shown various targets for use with an apparatus having a Scheiner component for use in collecting data for determining a myopic or hyperopic sphero-cylindrical refractive error of a human eye including cylinder axis and near addition, the Scheiner component comprising a pair of apertures that are spaced apart along a first axis.

Non-limiting examples of such Scheiner components are described above. The following description of various implementations of a target will be described in the context of a Scheiner component in the form of disc 102, including first aperture 104 and second aperture 106. The targets can take the place of any of the previously described targets, such as dot 202, including those presented on a physical substrate such as a printed planar card, and those presented on a display such as display 140, for example.

Turning to FIG. 27, there is shown a target 600 comprising a first feature for determining a correspondence between a presentation angle of the first feature and the first axis. In this and the subsequently described examples, the intention is to rotationally align the first feature and the first axis before determining the far point at various rotational angles.

In FIG. 27, the first feature takes the form of a straight line 602. As shown in FIG. 27, the presentation angle is 0°, relative to the vertical. At this presentation angle, a user would rotate the Scheiner component until there is correspondence between its axis and the straight line.

Alternatively, the Scheiner component can be kept stationary, and the target 600 rotated until there is correspondence between its axis and the straight line. This approach may be particularly applicable where the target 600 is presented on a display, and the angle of the target 600 can be accurately controlled. In this case, the Scheiner component may be rotatable between a number of known positions, such as those described above in relation to other implementations.

The straight line 602 is elongate. In other implementations, the first feature can take the form of one or more other elongate features. For example, FIG. 28 shows a first feature in the form of an isosceles triangle 604 that is elongated in the vertical direction. FIG. 29 shows a first feature in the form of a rectangle 606 that is elongated in the vertical direction. Both triangle 604 and rectangle 606 are solid shapes, although in other implementations, they can be line drawings, which may make them easier to rotationally align with the axis.

The first feature can comprise at least one linear component. That is, the first feature can include one more linear components along with other, non-linear components. For example, FIG. 30 shows a first feature in the form of a square 608. Square 608 includes two vertical sides and two horizontal sides, each of which is a linear component. Any of those sides can be used when aligning the first feature with the axis.

The target can includes a second feature for determining, using the Scheiner component of the apparatus, a first far point of the human eye by adjusting a distance between the human eye and the target. Such a target can take any suitable form, including that described above in relation to other implementations. For example, dot 202 or a different image can be used.

Alternatively, the second feature can include one or more linear or elongate features. Optionally, the first feature and the second feature are both elongate, in orthogonal directions relative to each other. For example, the square 608 of FIG. 30 incorporates both the first feature and the second feature, in the form of the vertical sides and horizontal sides, respectively.

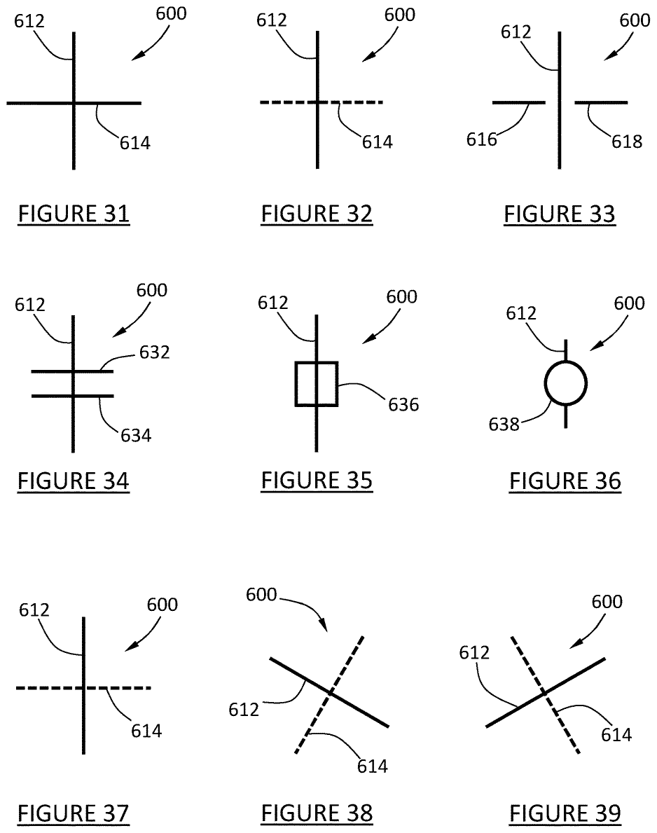

Turning to FIG. 31, there is shown a target 600 comprising a first feature in the form of a vertical line 612, and a second feature in the form of a horizontal line 614. In the illustrated fermentation, the vertical line 612 and horizontal line 614 are orthogonal to each other, and overlap to form a cross having equal length arms extending from a central point. Both vertical line 612 and horizontal line 614 are straight, solid lines.

The first and second features can be visually distinct from each other. For example, they can be of different colours, textures, thickness (where, for example, they take the form of lines), and the like. As shown in FIG. 32 for example, there is shown a target 600 that is similar to target 600 of FIG. 31, except that the horizontal line 614 is dashed. This can assist in distinguishing between the first and second features.

The use of target 600 of FIG. 32 will now be described with reference to FIGS. 37 to 45. For the purposes of this description, it will be assumed that apparatus 500 will be used, although any suitable apparatus comprising a Scheiner component can also be used.

Target 600 is initially presented with line 612 in the vertical position, and line 614 in the horizontal position, as shown in FIG. 37. Target 600 can be presented on a physical apparatus, such as a printed card, or on a display, such as display 140. Since line 612 is vertical, strip 446 of apparatus 500 is initially adjusted such that first apertures 404 are visible in window 434. The axis through first apertures 404 is vertical (relative to the user's eye). The intention is then to orientate the axis of first apertures 404 with line 612.

Looking through first apertures 404 at target 600, the user will see a representation of target 600 that depends upon the distance of their eye from target 600, any relative rotational offset between the axis of first apertures 404 and line 612, and the angle and amount of any myopic or hyperopic sphero-cylindrical refractive error of their eye.

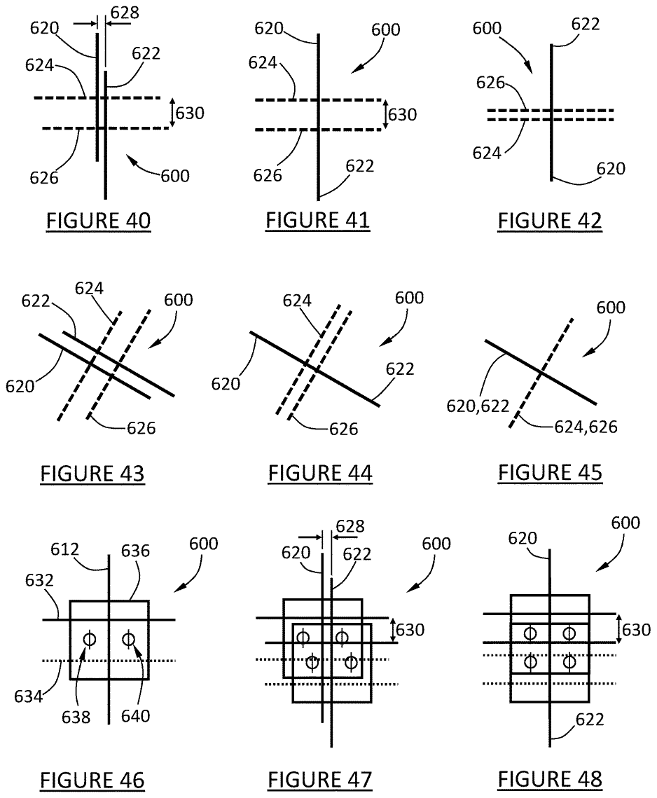

FIG. 40 shows one example of what one user might see upon looking through first apertures 404 at target 600. Target 600 appears to the user as a pair virtual images of target 600. That is, line 612 appears as virtual lines 620 and 622, and line 614 appears as virtual lines 624 and 626.

Virtual lines 620 and 622, and virtual lines 624 and 626, are offset from each other both horizontally and vertically. The horizontal offset 628, and part of the vertical offset 630, are a consequence of the axis of first apertures 404 not being rotationally aligned with line 612.

By slightly rotating the user's head while keeping apparatus 500 in position relative to the user's head, the virtual lines 620 and 622 can be brought into alignment. As shown in FIG. 41, this results in only a single vertical virtual line being visible, comprising the overlapped virtual lines 620 and 622. Once aligned in this way, the axis of first aperture 404 is rotationally aligned with line 612.

The residual vertical offset 630 in FIG. 41 is the combined result of the distance of the user's eye from target 600 and the amount of any myopic or hyperopic sphero-cylindrical refractive error of their eye at this particular angle.

The next step is to adjust the distance of the user's eye from target 600, while maintaining the virtual lines 620 and 622 in the overlapped position shown in FIG. 41. The user can be instructed to move closer to, and further away from, target 600 as needed, until the virtual lines 624 and 626 overlap exactly, resulting in an image corresponding to the original target 600 shown in FIG. 37. If the user goes too far in one direction, the virtual lines 624 and 626 can pass through and move away from each other, for example as shown in FIG. 42.

The distance between apparatus 500 and target 600 is then determined or estimated, and then recorded, for example in any of the ways described above.

Next, target 600 is presented at an angle offset from the initial presentation angle shown in FIG. 37. Although any offset angle can be used, greater accuracy may be achieved if the relative offset angles are maximised. For example, if the distance is determined or estimated for three rotational positions of the target, it may be desirable for each position to be offset by +/−60° relative to the other positions.

In FIG. 38, target 600 is presented at an angle that is offset by 60° counter-clockwise from that of FIG. 37. Strip 446 of apparatus 500 is adjusted such that the second apertures 406 are visible through window 434. The axis through second apertures 406 corresponds with the angle of target 600 in FIG. 38.

Looking through second apertures 406 at target 600, the user will see a different representation of target 600, depending upon the distance of their eye from target 600, any relative rotational offset between the axis of second aperture 406 and line 612, and the angle and amount of any myopic or hyperopic sphero-cylindrical refractive error of their eye.

FIG. 43 shows an example of what the user might see upon looking through second apertures 406 at target 600. Target 600 appears to the user as a pair virtual images of target 600, comprising virtual lines 620 and 622, and virtual lines 624 and 626.

Virtual line 620 and 622, and virtual lines 624 and 626, are offset from each other both horizontally and vertically (“horizontal” and “vertical” also being offset by 60° in the counter-clockwise direction in FIGS. 38 and 43). The horizontal offset 628, and part of the vertical offset 630, are a consequence of the axis of second aperture 404 not being rotationally aligned with line 612.

By slightly rotating the user's head or the apparatus 500, the virtual lines 620 and 622 can be brought into alignment. As shown in FIG. 42, this results in only a single “vertical” virtual line being visible, comprising the overlapped virtual lines 620 and 622. Once aligned in this way, the axis of second aperture 404 is rotationally aligned with line 612.

The residual vertical offset 630 in FIG. 44 is the combined result of the distance of the user's eye from target 600 and the amount of any myopic or hyperopic sphero-cylindrical refractive error of their eye at this particular angle.

The next step is to adjust the distance of the user's eye from target 600, while maintaining the virtual lines 620 and 622 in the overlapped position shown in FIG. 44. The user can be instructed to move closer to, and further away from, target 600 as needed, until the virtual lines 624 and 626 overlap exactly, resulting in an image corresponding to the original target 600 rotated by 60° in the counter-clockwise direction, as shown in FIG. 45.

The distance between apparatus 500 and target 600 is then determined or estimated, and then recorded, for example in any of the ways described above.

Next, target 600 is presented at an angle that is further offset from the initial presentation angle shown in FIG. 37. In FIG. 39, target 600 is presented at an angle that is offset by 60° clockwise from that of FIG. 37. Strip 446 is adjusted such that the third apertures 408 are visible through window 434. The axis through third apertures 408 corresponds with the angle of target 600 in FIG. 39.

Looking through third apertures 408 at target 600, the user will see a different representation of target 600, depending upon the distance of their eye from target 600, any relative rotational offset between the axis of third aperture 408 and line 612, and the angle and amount of any myopic or hyperopic sphero-cylindrical refractive error of their eye. The steps of the sequence illustrated in FIGS. 43-45 for the 60° counter-clockwise offset are then repeated for the 60° clockwise offset. The distance between apparatus 500 and target 600 is then determined or estimated, and then recorded, for example in any of the ways described above.

Once the distances for each of the orientations shown in FIGS. 37, 38, 39 are known, myopic or hyperopic sphero-cylindrical refractive error can be calculated, for example as described above.

Other implementations of a target comprising first features as defined herein can also be employed.

FIG. 33 shows a target 600 that is similar to target 600 of FIGS. 31 and 32, except that horizontal line 614 is replaced with a pair of horizontally spaced-apart horizontal lines 616 and 618. The space between horizontal lines 616 and 618 provide a clear region around vertical line 612, which some users may find easier when determining alignment as described above.

FIG. 34 shows a target 600 that is similar to target 600 of FIGS. 31 to 33, except that there are two vertically spaced-apart horizontal lines 632 and 634 overlapping vertical line 612. Some users may find two horizontal lines 632 and 634 easier to align when the user is adjusting the distance between the Scheiner component and the target 600.

FIG. 35 shows a target 600 comprising a square 636 overlaid on vertical line 612. Some users may find the use of a square (or other linear shape) easier to align when the user is adjusting the distance between the Scheiner component and the target 600.

FIG. 36 shows a target 600 comprising a circle 638 overlaid on vertical line 612. In this case, the vertical line to control does not continue through circle 638. Some users may find the use of a circle easier to align when the user is adjusting the distance between the Scheiner component and the target 600. Additionally, some users may find it easier to align the virtual vertical lines when they do not continue through the circle (or other shape, such as square 636 in FIG. 35).

FIG. 46 shows a target 600 comprising several different combinations of first and second features. Vertical line 612 passes through square 636, and first horizontal line 632 and second horizontal line 634 pass through both square 636 and vertical line 612. For clarity of illustration, second horizontal line 634 is shown as being dashed.

Target 600 of FIG. 46 also includes additional features in the form of a first circle 640 having a vertical line passing through it, and a second circle 644 having a vertical line passing through it. These additional features are disposed within the square 636 and between the first and second horizontal lines 632 and 634.

FIG. 47 shows one example of what one user might see upon looking through first apertures 404 at target 600 of FIG. 46. Target 600 appears to the user as a pair virtual images of target 600, which are offset from each other both horizontally and vertically. The horizontal offset 628, and part of the vertical offset 630, are a consequence of the axis of first apertures 404 not being rotationally aligned with line 612.

By slightly rotating the user's head or the apparatus 500, the virtual lines 620 and 622 can be brought into alignment. As shown in FIG. 48, this results in only a single vertical virtual line 612 being visible, comprising the overlapped virtual lines 620 and 622. Once aligned in this way, the axis of first aperture 404 is rotationally aligned with line 612.

The residual vertical offset 630 in FIG. 48 is the combined result of the distance of the user's eye from target 600 and the amount of any myopic or hyperopic sphero-cylindrical refractive error of their eye at this particular angle.

The next step is to adjust the distance of the user's eye from target 600, while maintaining the virtual lines 620 and 622 in the overlapped position shown in FIG. 48. The user can be instructed to move closer to, and further away from, target 600 as needed, until the various features of target 600 of FIG. 46 appear to overlap exactly, resulting in an image corresponding to the original target 600 shown in FIG. 46.

The distance between apparatus 500 and target 600 is then determined or estimated, and then recorded, for example in any of the ways described above. The process is then repeated at the offsets described above. Once the distances for the orientations of target 600 shown in FIGS. 37, 38, 39 are known, myopic or hyperopic sphero-cylindrical refractive error can be calculated, for example as described above.

Speaking generally, the second feature can comprise at least one linear component, which can comprise at least one second line for example.

In other implementations, the first and second features together comprise one or more crosses, grids, shapes, and/or sets of linear components.

Speaking generally, the target can be rotatable. The rotation can be continuous, or can be between first and second orthogonal rotational positions, between first, second, and third rotational positions, and between any greater number of rotational positions. Optionally, the rotational positions can be equidistant from each other. The rotational positions can optionally be indicated by way of indicia, and/or by way of feedback. For example, where the target is presented on a physical surface such as a dial, detents can be used to indicate when the target is at each intended rotational position, as describe above in relation to other implementations.

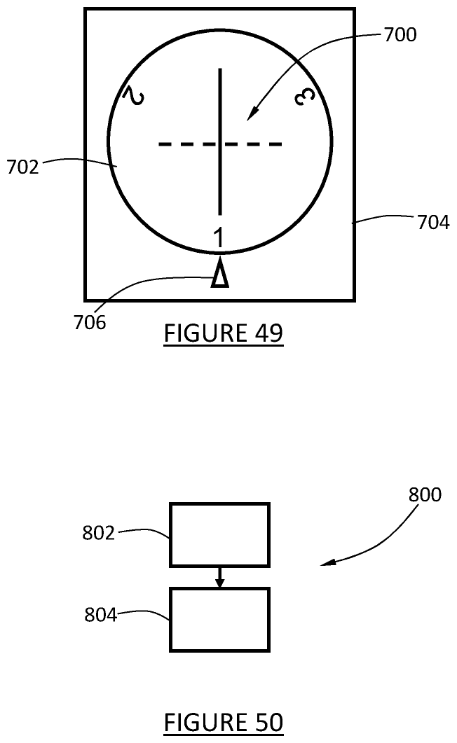

FIG. 49 shows an example of a target 700, which is printed onto a dial 702 that is mounted for rotation on a frame 704. Indicia are provided in the form of the numbers 1, 2, and 3 printed on the face of the dial 702, and a marker 706 printed on frame 704 adjacent to the edge of dial 702. The rotational position of the target 700 can be established by rotating the dial 702 such that the required number is aligned with the marker 706. In other implementations, the numbers (or other indicia) can be on the frame 704, and the marker 706 can be on the dial 702.

In yet other implementations, a target can be provided without the first feature (e.g., any of the targets of FIGS. 27 to 50 can be provided without a vertical line or other elongate shape). In that case, rotational correspondence can be established based on the user's own observations, feedback from a clinician or other operator, or feedback based on measurements made by, e.g., any system involved in the testing. For example, image capture can be used to ensure that the user's head and the Scheiner component are appropriately aligned.

In yet other implementations, the target can be used with a method such as method 146. A rotational angle of a first cylindrical axis of an eye for which data is to be collected is determined. As described above, the skilled person will appreciate that the first cylindrical axis may correspond with either the maximum or minimum refractive power of the eye being measured but, as the skilled person will understand, it is possible to calculate and represent the sphero-cylindrical refractive error in two ways (known as the positive cylinder or the minus cylinder).

The rotational angle can be measured in any suitable way, such as any of those described above. The user can then be instructed to orientate disc 102 based on the determined rotational angle. If the rotational angle was measured, for example by way of the user's interaction with clock target 150, that information can be used to determine an appropriate rotational position of disc 102 during the step of determining 152 the first far point.

Since the rotational angle of a first cylindrical axis of an eye for which data is to be collected has been determined, it is then only necessary to measure the far point at two angles as described above in relation to FIG. 26, rather than the three angles described above in relation to the various implementations of FIGS. 37 to 49. Correspondence between a presentation angle of the first feature and the first axis can be determined for both angles, as described above.

Turning to FIG. 50, there is shown a method 800 of collecting data for determining a sphero-cylindrical refractive error of a human eye. The method comprises presenting 802 a target to a user, at at least two different presentation angles. The target is, for example, as described herein and/or as defined in the claims.

Next, the method comprises providing 804 an instruction to the user to adjust, for each of the presentation angles, a rotational angle of:
their eye about an axis between their eye and the target and while looking through the Scheiner component; and/or
the current presentation angle;
such that there is a visual indication of correspondence between the angle of the first feature and the first axis.

The visual indication can comprise the alignment of two virtual images of the first feature visible to the user.

The visual indication can comprise the overlapping of the two virtual images of the first feature visible to the user.

The method can optionally comprise presenting the target to the user at two orthogonal angles, or at three or more angles. The angles can optionally be equidistant.

Some or all of the steps of the methods described herein can be computer-implemented, for example using the computer hardware described herein, running software for implementing the steps to be performed by the computer.

The invention has been described with reference to a preferred and other embodiments. However, it will be appreciated that variations and modifications can be effected by a person of ordinary skill in the art without departing from the scope of the invention.

1. An apparatus for use in collecting data for determining a myopic or hyperopic sphero-cylindrical refractive error of a human eye including cylinder axis and near addition, the apparatus comprising:
a Scheiner component comprising first and second spaced apart apertures;
orientation indicia on or adjacent to the Scheiner component for allowing a rotational position of the apertures relative to a user's eye to be determined when the apparatus is in use.

2. The apparatus as claimed in claim 1, wherein the orientation indicia comprises rotational indicia on the Scheiner component.

3. The apparatus as claimed in claim 1 or claim 2, comprising a frame, the Scheiner component being mounted for rotation relative to the frame.

4. The apparatus as claimed in claim 1 or claim 2, wherein the Scheiner component comprises at least first, second, and third pairs of the first and second spaced apart apertures, the first, second, and third pairs being spaced apart from each other on the Scheiner component.

5. The apparatus as claimed in claim 4, configured for use with the Scheiner component in each of a plurality of positions, each of the positions corresponding with one of the at least first, second, and third pairs of apertures being in a viewing position, wherein, when at the viewing position, each of the first, second, and third pairs of the apertures has its first and second apertures aligned at a different angle as compared with an angle of alignment of the other of the first, second, and third pairs at the viewing position.

6. The apparatus as claimed in claim 5, comprising a frame, the Scheiner component being mounted to the frame for movement between each of the plurality of positions.

7. The apparatus as claimed in claim 6, the Scheiner component being mounted to the frame for rotation between each of the plurality of positions.

8. The apparatus as claimed in claim 6, the Scheiner component being mounted to the frame for translational movement between each of the plurality of positions.

9. The apparatus as claimed in any one of claims 6 to 8, wherein the frame comprises a window defining the viewing position, the apparatus being configured such at each of the plurality of positions, a different one of the first, second, and third pairs appears through the window.

10. The apparatus as claimed in claim 3 or any one of claims 6 to 9, wherein the orientation indicia comprises frame indicia on the frame.

11. The apparatus as claimed in claim 10, wherein the frame indicia comprises a scale.

12. The apparatus as claimed in claim 11, wherein the scale is configured for allowing a distance from an image capture device to the frame to be determined by a processor in communication with the image capture device.

13. The apparatus as claimed in any one of claims 9 to 12, wherein the frame comprises a handle that can be grasped to hold the apparatus in front of a user's eye when the apparatus is in use.

14. The apparatus as claimed in any one of claims 9 to 12, wherein the frame comprises a bridge-engaging portion for engaging a bridge of a user's nose when the apparatus is in use.

15. The apparatus as claimed in any one of the preceding claim, comprising a positive power lens for generating a positive refractive offset.

16. The apparatus as claimed in claim 15, wherein the positive power lens is removably mountable to the apparatus.

17. The apparatus as claimed in claim 15 or 16, wherein the apparatus comprises a lens mount for mounting the lens.

18. The apparatus as claimed in claim 17, wherein the lens mount is moveable between a first position in which the lens is in front of a user's eye when the apparatus is in use, and a second position in which the lens is not in front of the user's eye when the apparatus is in use.

19. The apparatus as claimed in claim 18, wherein the lens mount is mounted such that it can be moved between the first position and the second position.

20. A target for use with an apparatus having a Scheiner component for use in collecting data for determining a myopic or hyperopic sphero-cylindrical refractive error of a human eye including cylinder axis and near addition, the Scheiner component comprising a pair of apertures that are spaced apart along a first axis, the target comprising a first feature for determining a correspondence between a presentation angle of the first feature and the first axis.

21. The target of claim 20, wherein the first feature is elongate.

22. The target of claim 21, wherein the first feature comprises at least one linear component.

23. The target of claim 22, wherein the linear component comprises a first line.

24. The target of any one of claims 20 to 23, wherein the target includes a second feature for determining, using the Scheiner component of the apparatus, a first far point of the human eye by adjusting a distance between the human eye and the target.

25. The target of claim 24, wherein the first feature and the second feature are elongate in orthogonal directions relative to each other.

26. The target of claim 25, wherein the second feature comprises at least one linear component.

27. The target of claim 27, wherein the linear component comprises at least one second line.

28. The target of any one of claims 20 to 27, wherein the first and second features together comprise one or more crosses, grids, shapes, and/or sets of linear components.

29. The target of claim 28, wherein:
the first feature comprises at least one line; and
the second feature comprises a line extending orthogonally to the line of the first feature.

30. The target of claim 29, wherein the first feature and the second feature are visually distinct from each other.

31. The target of any one of claims 20 to 30, wherein the target is rotatable.

32. The target of claim 31, wherein the target is rotatable between first and second orthogonal rotational positions.

33. The target of claim 31, wherein the target is rotatable between first, second, and third equidistant rotational positions.

34. The target of any one of claims 31 to 33, comprising indicia for determining a rotational position of the target.

35. A method of collecting data for determining a sphero-cylindrical refractive error of a human eye, the method comprising:
determining a rotational angle of a first cylindrical axis of an eye for which data is to be collected;
determining, based on the Scheiner principle, a first far point of the eye for the first cylindrical axis using the apparatus of any one of claims 1 to 19, and/or the target of any one of claims 20 to 34; and
determining, based on the Scheiner principle, a second far point of the eye for a second cylindrical axis using the apparatus and/or the target, with the Scheiner component orientated orthogonally to an angle at which the first far point was determined, wherein a rotational angle of the second cylindrical axis is orthogonal to the rotational angle of the first cylindrical axis.

36. A method of collecting data for determining a sphero-cylindrical refractive error of a human eye, using the apparatus of any one of claims 5 to 9, or any one of claims 10 to 19 when dependent upon any one of claims 5 to 9, the method comprising:
positioning the first pair of apertures at the viewing position;
determining, based on the Scheiner principle, a first far point of the human eye for the first pair of first and second apertures by adjusting a distance between the human eye and a target;
positioning the second pair of apertures at the viewing position;
determining, based on the Scheiner principle, a second far point of the human eye for the second pair of first and second apertures at the second position, by adjusting a distance between the human eye and a target;
positioning the third pair of apertures at the viewing position; and
determining, based on the Scheiner principle, a third far point of the human eye for the third pair of first and second apertures at the third position, by adjusting a distance between the human eye and a target.

37. The method as claimed in claim 35 or claim 36, comprising:
determining whether a refractive error of an eye for which data is to be collected is greater than a predetermined value; and
responsive to determining that the refractive error of the eye is greater than the predetermined value, positioning a positive power lens relative to the apparatus such that it is in front of a user's eye when the apparatus is in use, prior to determining the first and second far points.

38. The method as claimed in claim 37, wherein the predetermined value is less than 0.0 D.

39. The method as claimed in claim 37, wherein the predetermined value is less than −1.00 D.

40. The method as claimed in any one of claims 37 to 39, wherein positioning the positive power lens comprises installing the lens on a lens mount of the apparatus.

41. The method as claimed in any one of claims 37 to 40, wherein positioning the positive power lens comprises moving the positive power lens from a second position in which the lens is not in front of the user's eye when the apparatus is in use, to a first position in which the lens is in front of a user's eye when the apparatus is in use.

42. The method as claimed in any one of claims 35 to 41, performed using the target of any one of claims 20 to 34.

43. A method of collecting data for determining a sphero-cylindrical refractive error of a human eye, the method comprising:
presenting the target of any one of claims 20 to 34 to a user, at at least two different presentation angles;
providing an instruction to, or causing, the user to adjust, for each of the presentation angles, a rotational angle of:
their eye about an axis between their eye and the target and while looking through the Scheiner component; and/or
the current presentation angle;
such that there is a visual indication of correspondence between the angle of the first feature and the first axis.

44. The method of claim 43, wherein the visual indication comprises the alignment of two virtual images of the first feature visible to the user.

45. The method of claim 44, wherein the visual indication comprises the overlapping of the two virtual images of the first feature visible to the user.

46. The method of any one of claims 43 to 45, comprising presenting the target to the user at two orthogonal angles.

47. The method of any one of claims 43 to 46, comprising presenting the target to the user at three or more angles.

48. The method of claim 47, comprising presenting the target to the user at exactly three equidistant angles.
